(19) United States
(12) Patent Application Publication
(10) Pub. No.: US 2011/0059038 A1
(43) Pub. Date: Mar. 10, 2011

(54) METHOD OF STABILIZING A POLYSILOXANE EMULSION AND COMPOSITIONS CONTAINING STABILIZED EMULSIONS

(76) Inventors: Stephanie E. Gabelnick, Aurora, IL (US); Minoru Hosokawa, Kanagawa-ken (JP); Atsushi Yokomaku, Chiba-ken (JP); Toshiyuki Kashiwai, Chiba-ken (JP)

(21) Appl. No.: 12/948,239

(22) Filed: Nov. 17, 2010

Related U.S. Application Data
(63) Continuation of application No. 11/831,858, filed on Jul. 31, 2007.

(51) Int. Cl. A61K 31/80; A61Q 19/00

(52) U.S. Cl. 424/78.03

(57) ABSTRACT

A method of stabilizing an emulsion containing a polysiloxane and one or more surfactants comprising adding an effective amount of dispersion containing a DADMAC/acrylamide copolymer and one or more sulfate salts into said emulsion is disclosed. An emulsion/cosmetically acceptable emulsion comprising a polysiloxane, at least one surfactant, and a dispersion containing DADMAC/acrylamide copolymer and one or more sulfate salts is also disclosed.

### CROSS REFERENCE TO RELATED APPLICATIONS

The application is a continuation application of U.S. application Ser. No. 11/831,858, “METHOD OF STABILIZING A POLYSILOXANE EMULSION AND COMPOSITIONS CONTAINING STABILIZED EMULSIONS,” filed Jul. 31, 2007, now pending.

### FIELD OF THE INVENTION

The invention pertains to a method of stabilizing an emulsion containing polysiloxane and one or more surfactants, as well as emulsions/cosmetically acceptable emulsions containing polysiloxane and one or more surfactants.

### BACKGROUND

Emulsions containing polysiloxane and one or more surfactants are not stable. More specifically, the surfactant/polysiloxane emulsion is not stable, and separates after a period of time. This dilemma is especially problematic in the consumer hair care market where delivering a stable emulsion containing polysiloxane and surfactants would be desirable. Therefore, there is a need for a composition that comprises a stable emulsion, which contains polysiloxane and one or more surfactants.

### SUMMARY OF THE INVENTION

The present invention provides for a method of stabilizing an emulsion containing a polysiloxane and one or more surfactants comprising: adding an effective amount of dispersion containing a DADMAC/acrylamide copolymer and one or more sulfate salts into said emulsion.

The present invention also provides for a cosmetically acceptable emulsion comprising a polysiloxane, at least one surfactant, and a dispersion containing DADMAC/acrylamide copolymer and one or more sulfate salts.

The present also provides for an emulsion comprising a polysiloxane containing emulsion, wherein said composition comprises a polysiloxane, at least one surfactant, and a dispersion containing DADMAC/acrylamide copolymer and one or more sulfate salts.

### DETAILED DESCRIPTION OF THE INVENTION

A DADMAC/acrylamide copolymer is a copolymer containing polydiallyldimethyl ammonium chloride and acrylamide, as well as derivatives thereof.

A dispersion containing DADMAC/acrylamide and one or more sulfate salts is available from Nalco Company, Naperville, Ill., as Ultimer® CG-600.

One or more types of surfactants may be present in a composition containing a polysiloxane/surfactant containing emulsion.

Anionic surfactants useful herein include those disclosed in U.S. Pat. No. 5,573,709, incorporated herein by reference. Examples include alkyl and alkyl ether sulfates. Specific examples of alkyl ether sulfates which may be used in this invention are sodium and ammonium salts of lauryl sulfate, lauryl ether sulfate, coconut alkyl triethylene glycol ether sulfate, tallow alkyl triethylene glycol ether sulfate, and tallow alkyl hexaoxyethylene sulfate. Preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surfactants is the alkyl sulfuric acid salts. Important examples are the salts of an organic sulfuric acid reaction product of a hydrocarbon of the methane series, including iso-, neo-, ineso-, and n-paraffins, having about 8 to about 24 carbon atoms, preferably about 12 to about 18 carbon atoms and a sulfonating agent, e.g., SO₃, H₂SO₄, oleum, obtained according to known sulfonation methods, including bleaching and hydrolysis. Preferred are alkali metal and ammonium sulfated C₁₂₋₃₈ n-paraffins.

Additional synthetic anionic surfactants include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinanrate, tetrasodium N-(1,2-dicarboxyethyl)-N-octadecylsulfosuccinamate, diamyl ester of sodium sulfosuccinic acid, dihexyl ester of sodium sulfosuccinic acid, and dioctyl esters of sodium sulfosuccinic acid.

Preferred anionic surfactants for use in the composition of this invention include ammonium lauryl sulfate, ammonium laureth sulfate, trlethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethano famine lauryl sulfate, sodium tridecyl benzene sulfonate, and sodium dodecyl benzene sulfonate.

Amphoteric surfactants which may be used in the composition of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOLTM as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Cationic surfactants generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof. The structure or representative quaternary ammonium compounds is provided in the CTFA Cosmetic Ingredient Handbook, Fifth Edition, 1988, page 40.

Examples of quaternary ammonium compounds include but are not limited to: behentrimonium chloride, cocotrimonium chloride, cethethyldimonium bromide, dibehenyldimonium chloride, dihydrogenated tallow benzylmonium chloride, disoyadimonium chloride, ditallowdimonium chloride, hydroxycetyl hydroxyethyl dimonium chloride, hydroxyethyl behenamidopropyl dimonium chloride, hydroxyethyl cetyldimonium chloride, hydroxyethyl tallowdimonium chloride, myristalkonium chloride, PEG-2 oleamonium chloride, PEG-5 stearmonium chloride, PEG-15 cocoyl quaternium 4, PEG-2 stearalkonium 4, lauryltrimonium chloride; Quatemium-16; Quatemium-18, lauralkonium chloride, olealkmonium chloride, cetylpyridinium chloride, Polyquaternium-5, Polyquaternium-6, Polyquatemium-7, Polyquatemium-10, Polyquaternium-22, Polyquatemium-37, Polyquaternium-39, Polyquaternium-47, polyquaternium-55, cetyl trimonium chloride, dilauryldimonium chloride, cetalkonium chloride, dicetyldimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The compositions may include di-long chain amines from about C₁₀ to C₂₂, long chain fatty amines from about C₁₀ to C₂₂, and mixtures thereof. Specific examples include dipalmitylamine, laurarnidopropyldimethyl, stearamidopropyl dimethylamine.

The compositions of this invention may also include fatty alcohols (typically monohydric alcohols), ethoxylated fatty alcohols, and di-tail phospholipids, which can be used to stabilize emulsion or dispersion forms of the compositions. They also provide an acceptable viscosity. Selection of the fatty alcohol is not critical, although those alcohols characterized as having fatty chains of C₁₀ to C₃₂, preferably C₁₄ to C₂₂, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used, the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof. Phospholipids such as phosphatidylserine and phosphatidylcholine, and mixtures thereof may also be included.

Nonionic surfactants, which can be used in the composition of this invention include those broadly defined as compounds produced by the condensation of alkylene oxide groups (hydrophilic in nature) with an organic hydrophobic compound, which may be aliphatic or alkyl aromatic in nature. Examples of preferred classes of nonionic surfactants are: the long chain alkanolarnides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surfactants such as betaines can also be useful in the composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-arboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like, amidobetaines and amidosulfobetaines, wherein the RCONH(CH₂)₃ radical is attached to the nitrogen atom of the betaine are also useful in this invention.

The anionic, cationic, nonionic, amphoteric or zwitterionic surfactants used in the composition of this invention are typically used in an amount from about 0.1 to 50 percent by weight, preferably from about 0.5 to about 40 percent by weight, more preferably from about 1 to about 20 percent by weight.

Emulsions may be formed by various means that may be appreciated by one of ordinary skill in the art. For example, emulsions may be formed by stirring a solution containing one or more components, and by other modes of agitation.

As stated above, the present invention provides for a method of stabilizing an emulsion containing a polysiloxane and one or more surfactants comprising: adding an effective amount of composition containing a DADMAC/acrylamide copolymer and one or more sulfate salts into said emulsion.

In one embodiment, a method of stabilizing a polysiloxane emulsion entails adding a surfactant and optionally other additives, followed by DADMAC/acrylamide copolymer, and followed by polysiloxane, which in it of itself is added as an emulsion.

In another embodiment the polysiloxane is a polydimethylsiloxane.

In another embodiment, the copolymer of DADMAC/acrylamide contains from about 70% moles of acrylamide and about 30% moles of DADMAC.

In another embodiment, the DADMAC/acrylamide copolymer has a weight average molecular weight of from about 1,000,000 to about 4,000,000.

In another embodiment, the effective amount of DADMAC/acrylamide copolymer is from about 0.01% to about 1% weight based upon the amount of polymer actives in the composition.

In another embodiment, the ratio between surfactant and copolymer in the composition is from about 18:1 to about 50:1.

In another embodiment, the surfactant in said composition is selected from the group consisting of: sodium lauryl ether sulfate, sodium lauryl sulfate, cocamidopropyl betaine, and a combination thereof.

In a further embodiment, the sulfate salts are ammonium sulfate, sodium sulfate, or a combination thereof.

In another embodiment, the emulsion is used to treat hair, skin and other external body treatments.

As stated above, the present invention also provides for an emulsion/cosmetically acceptable emulsion containing a polysiloxane, at least one surfactant, and a dispersion containing DADMAC/acrylamide copolymer and one or more sulfate salts.

In one embodiment, the emulsion is prepared by adding a surfactant and optionally other additives, followed by DADMAC/acrylamide copolymer, and followed by polysiloxane, which in it of itself is added as an emulsion.

In another embodiment, the polysiloxane is a polydimethylsiloxane.

In another embodiment, the copolymer has from about 70% moles of acrylamide and about 30% moles of DADMAC.

In another embodiment, the DADMAC/acrylamide copolymer has a weight average molecular weight of from about 1,000,000 to about 4,000,000.

In another embodiment, the effective amount is about 0.01% to about 1% weight based upon the amount of polymer actives in the composition.

In another embodiment, the ratio between surfactant and copolymer is about 18:1 to about 50:1.

In another embodiment, the surfactant is selected from the group consisting of: sodium lauryl ether sulfate, sodium lauryl sulfate, cocamidopropyl betaine, and a combination thereof.

In another embodiment, the sulfate salts are ammonium sulfate, sodium sulfate, or a combination thereof.

In another embodiment, the emulsion/cosmetically acceptable emulsion further comprises one or more cosmetically acceptable excipients.

In another embodiment, the cosmetically acceptable excipients are selected from the group consisting of: water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain, amines from about C₁₀ to C₂₂, long chain fatty amines from about C₁₀ to C₂₂, fatty alcohols, ethoxylated fatty alcohols, and di-tail phospholipids.

In another embodiment, the emulsion/cosmetically acceptable emulsion is a hair cleaner/shampoo.

1. A cosmetically acceptable emulsion comprising a polysiloxane, at least one surfactant, and a dispersion containing DADMAC/acrylamide copolymer and one or more sulfate salts.

2. The cosmetically acceptable emulsion of claim 1, further comprising one or more cosmetically acceptable excipients.

3. The cosmetically acceptable emulsion of claim 2, wherein the excipients are selected from the group consisting of: water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C₁₀ to C₂₂, long chain fatty amines from about C₁₀ to C₂₂, fatty alcohols, ethoxylated fatty alcohols, and di-tail phospholipids.

4. The cosmetically acceptable emulsion of claim 1, wherein said copolymer has from about 70% moles of acrylamide and about 30% moles of DADMAC.

5. The cosmetically acceptable emulsion of claim 1, wherein said DADMAC/acrylamide copolymer has a weight average molecular weight of from about 1,000,000 to about 4,000,000.

6. The cosmetically acceptable emulsion of claim 1, wherein the ratio between the surfactant and the copolymer is from about 18:1 to about 50:1.

7. The cosmetically acceptable emulsion of claim 1, wherein the surfactant is selected from the group consisting of: sodium lauryl ether sulfate, sodium lauryl ether sulfate, sodium lauryl sulfate, cocamidopropyl betaine, and a combination thereof.

8. The cosmetically acceptable emulsion of claim 1, wherein said sulfate salts are ammonium sulfate, sodium sulfate, or a combination thereof.

9. The cosmetically acceptable emulsion of claim 1, wherein said polysiloxane is a polydimethylsiloxane.

10. An emulsion comprising a polysiloxane, at least one surfactant, and a dispersion containing a DADMAC/acrylamide copolymer and one or more sulfate salts.

11. A method of stabilizing the emulsion of claim 10, the method comprising adding an effective amount of the dispersion containing the DADMAC/acrylamide copolymer and the one or more sulfate salts into said emulsion.

12. The method of claim 11, wherein said copolymer has from about 70% moles of acrylamide and about 30% moles of DADMAC.

13. The method of claim 11, wherein said DADMAC/acrylamide copolymer has a weight average molecular weight of from about 1,000,000 to about 4,000,000.

14. The method of claim 11, wherein said effective amount is from about 0.01% to about 1% weight based upon the amount of polymer actives in the composition.

15. The method of claim 11, wherein the ratio between surfactant and copolymer is about 18:1 to about 50:1.

16. The method of claim 11, wherein the surfactant is selected from the group consisting of: sodium lauryl ether sulfate, sodium lauryl ether sulfate, sodium lauryl sulfate, cocamidopropyl betaine, and a combination thereof.

17. The method of claim 11, wherein said sulfate salts are ammonium sulfate, sodium sulfate, or a combination thereof.

18. The method of claim 11, wherein said emulsion is used to treat hair, skin and other external body treatments.

19. The method of claim 11, wherein said polysiloxane is a polydimethylsiloxane.

20. The method of claim 11, wherein said effective amount is about 0.01% to about 1% weight based upon the amount of polymer actives in the composition.
